SEQUENCE LISTING (3 sequences)

SEQ ID NO: 1 - DNA, 1794 bp, Streptomyces roseosporus L30
gtgagtgagagccgctgtgccgggcagggcctggtgggggcactgcggacctgggcacggacacgtgcccgggagactgccgtggttctcgtacgggacaccggaaccaccgacgacacggcgtcggtggactacggacagctggacgagtgggccagaagcatcgcggtgaccctccgacagcaactcgcgccggggggacgggcacttctgctgctgccgtccggcccggagttcacggccgcgtacctcggctgcctgtacgcgggtctggccgccgtaccggcgccgctgcccggggggcgccacttcgaacgccgccgtgtcgcggccatcgccgccgacagcggagccggcgtggtgctgaccgtcgcgggtgagaccgcctccgtccacgactggctgaccgagaccacggccccggctactcgcgtcgtggccgtggacgaccgggcggcgctcggcgacccggcgcagtgggacgacccgggcgtcgcgcccgacgacgtggctctcatccagtacacctcgggctcgaccggcaaccccaagggcgtggtcgtgacccacgccaacctgctggcgaacgcgcggaatctcgccgaggcctgcgagctgaccgccgccactcccatgggcggctggctgcccatgtaccacgacatggggctcctgggcacgctgacaccggccctgtacctcggcaccacgtgcgtgctgatgagctccacggcattcatcaaacggccgcacctgtggctacggaccatcgaccggttcggcctggtctggtcgtcggctcccgacttcgcgtacgacatgtgtctgaagcgcgtcaccgacgagcagatcgccgggctggacctgtcccgctggcggtgggccggcaacggcgcggagcccatccgggcagccaccgtacgggccttcggcgaacggttcgcccggtacggcctgcgccccgaggcgctcaccgccggctacgggctggccgaggccaccctgttcgtgtcgaggtcgcaggggctgcacacggcacgagtcgccaccgccgccctcgaacgccacgaattccgcctcgccgtacccggcgaggcagcccgggagatcgtcagctgcggtcccgtcggccacttccgcgcccgcatcgtcgaacccggcgggcaccgtgttctgccgcccggccaggtcggcgagctggtcctccagggagccgccgtctgcgccggctactggcaggccaaggaggagaccgagcagaccttcggcctcaccctcgacggcgaggacggtcactggctgcgcaccggcgatctcgccgccctgcacgaagggaatctccacatcaccggccgctgcaaagaggccctggtgatacgaggacgcaatctgtacccgcaggacatcgagcacgaactccgcctgcaacacccggaacttgagagcgtcggcgccgcgttcaccgtcccggcggcacctggcacgccgggcttgatggtggtccacgaagtccgcaccccggtccccgccgacgaccacccggccctggtcagcgccctgcgggggacgatcaaccgcgaattcggactcgacgcccagggcatcgccctggtgagccgcggcaccgtactgcgtaccaccagcggcaaggtccgccggggcgccatgcgtgacctctgcctccgcggggagctgaacatcgtccacgcggacaagggctggcacgccatcgccggcacggccggagaggacatcgcccccactgaccacgctccacatccgcaccccgcgtaa

SEQ ID NO: 2 - DNA, 407 bp, Streptomyces roseosporus L30
  misc_feature (4)..(4): n is a, t, g, or c
  misc_feature (8)..(8): n is a, t, g or c
  misc_feature (9)..(9): n is a, t, g or c
  misc_feature (13)..(13): n is a, t, g or c
  misc_feature (16)..(16): n is a, t, g or c
  misc_feature (17)..(17): n is a, t, g or c
catnattnncatntanncccctccccaccacctgcccagtgtgacgtttgcgcagatgagaacgtgcgtaaacgccgcatacgcaaagatcgtccctgccgggacccattgacgttcgcaggggcgtggaacatactggcgatcaagtcgcacaggaaccaacaggcacaccaaccacaggcgttacagggggggttggtgtttcgtccatatcaagtggtttggtccgccgaagcggttggacctcacatgacggcaacagggcattcgcacatgcctgatgacgggacggcacacctcacgcagcggcgaccggtcgcaagccggacgcggaatgactccctgccttacaggtatgcgagcgcggatgcgtcgttcgaccggagtcaggagggggagtgcctgcc

SEQ ID NO: 3 - DNA, 213 bp, Streptomyces roseosporus L30
atggctgctggcagcgagaggcctctcaacgaggtcaagtttctgaccgtggcggaagtcgcctcggtcatgcgagtgtcgaagatgacggtgtaccgcttggtgcacagcggtcatctgccggcgatccgggtgggcaggtccttccgggtgccggagcaagcggttcacgcgtatctccgcgagtcgttcgtgggggtggaatcagcctga

(19) United States
(12) Patent Application Publication
(10) Pub. No.: US 2021/0079487 A1
(43) Pub. Date: Mar. 18, 2021

(54) GENETIC SCREENING METHOD OF NEGATIVE REGULATORY FACTORS OF STREPTOMYCES BIOSYNTHESIS GENE CLUSTER

(71) Applicant: ZHEJIANG UNIVERSITY, Hangzhou City, Ahejiang Province (CN)

(72) Inventors: Yongquan LI, Hangzhou City, Zhejiang Province (CN); Xuming MAO, Hangzhou City, Zhejiang Province (CN); Shuai LUO, Hangzhou City, Zhejiang Province (CN)

(21) Appl. No.: 17/042,170

(22) PCT Filed: Mar. 20, 2019
(86) PCT No.: PCT/CN2019/078798

§ 371 Date: Sep. 27, 2020

(30) Foreign Application Priority Data
May 10, 2018 (CN) 201810443289.2

(51) Int. Cl. C12Q 1/6897; C12N 15/76

(52) U.S. Cl. CPC C12Q 1/6897; C12N 15/76

(57) ABSTRACT

The present invention provides a screening method of negative regulatory factors of a Streptomyces biosynthesis gene cluster, the method including: constructing a reporter system in a Streptomyces cell, which is mediated by a promoter of a self-owned target gene of the Streptomyces cell, and then randomly mutating Streptomyces with the reporter system by using a random mutation system constructed based on a transposon Himar1; intensively screening Streptomyces strains that have been subjected to random mutation to obtain a Streptomyces strain with high expression of the target gene; performing phage packaging on a genome of the Streptomyces strain with high expression of the target gene and screening out a cosmid with a random insert; and determining the position of the random insert in the genome of the Streptomyces strain with high expression of the target gene by sequencing DNAs of the cosmid.

### TECHNICAL FIELD

The present invention relates to the field of biochemistry and molecular biology, and in particular to a new genetic screening method of negative regulatory factors of a Streptomyces biosynthesis gene cluster.

### BACKGROUND

Over the past few centuries, scientists have isolated and screened tens of thousands of natural products from nature. However, in recent decades, it is increasingly difficult for scientists to discover and separate new natural products. Genomics, which has flourished since the beginning of this century, has pointed out a new direction for us to discover new natural products. Many proteins play a regulatory role in organisms, among which a class of regulatory proteins can participate in the regulation of gene expression, which can activate or inhibit the transcription level of specific genes. Researchers try to find out and isolate more interesting compounds by looking for specific regulatory factors, activating and overexpressing new secondary metabolic gene clusters in organisms. However, a simple, accurate and efficient molecular biological means is urgently needed to find the pathway-specific regulatory factors of silenced gene clusters, so as to further activate and highly express the synthesis gene clusters of new compounds that are silenced or inhibited in vivo.

As a powerful tool for genome modification, transposons are widely used in the genetic modification of eukaryotic cells. Transposons can be randomly or specifically inserted into a certain position in the genome, thus affecting the subsequent transcription and translation process of genes at this position, resulting in the functional loss of target genes in organisms.

Streptomyces belongs to actinomycetes and is a Gram-positive bacterium. Streptomyces has a complex life cycle, including morphological differentiation from substrate mycelium, aerial mycelium to spores. At different stages of the life cycle of Streptomyces, the regulatory proteins in Streptomyces regulate these life cycles smoothly and orderly by activating or inhibiting the transcription levels of various genes. Most antibiotics used in the world are produced by Streptomyces secondary metabolism. Therefore, it is of great significance to explore and study the pathway specific regulatory factors of a secondary metabolite synthesis gene cluster in Streptomyces both in basic scientific research and in industrial production.

The regulatory effect of the regulatory protein on the target gene cluster is regulating the promoter activity of the target gene cluster. Based on the above, we invented a new genetic screening method for unknown negative regulatory factors of the Streptomyces biosynthesis gene cluster in vivo. The method is efficient, accurate and easy to operate, and provides a new research method for screening the regulatory factors of a target gene cluster in Streptomyces.

### SUMMARY

In view of researches on the genetic screening of regulatory proteins of in vivo gene clusters of Streptomyces, the present invention aims to provide a genetic screening method of negative regulatory factors of a Streptomyces biosynthesis gene cluster, which is a new method for in vivo screening of regulatory proteins.

In the present invention, a reporter system mediated by a promoter of a self-owned target gene of a Streptomyces cell is constructed in the Streptomyces cell, and then Streptomyces with the reporter system is randomly mutated by using a random mutation system constructed based on a transposon Himar1. Streptomyces strains that have been subjected to random mutation are intensively screened to obtain a Streptomyces strain with high expression of the target gene. In a fourth step, a genome of the Streptomyces strain with high expression of the target gene is packaged by a phage and a cosmid with a random insert is screened out. Finally, a position of the random insert in the genome of the Streptomyces strain with high expression of the target gene is determined by sequencing DNAs of the cosmid. The specific steps are as follows:

(1) selecting a target gene, which needs to be screened for a regulatory factor, in a Streptomyces genome, and amplifying an upstream promoter sequence of the target gene;

(2) selecting an available reporter gene system in the Streptomyces, constructing a plasmid system in which the reporter gene system can be genetically operated in the Streptomyces, and determining that there is no promoter upstream of a reporter gene in the plasmid system;

(3) integrating the promoter sequence in step (1) into a reporter plasmid system in step (2) upstream of the reporter gene;

(4) transducting a reporter plasmid obtained in step (3) into wild-type Streptomyces by conjugation, and verifying;

(5) according to the selected reporter gene system, performing threshold screening of an expression level of the reporter gene of the Streptomyces strain obtained in the step (4);

(6) amplifying three DNA fragments: 10 hygromycin resistance gene hph; thiostrepton-induced promoter and transposon tipAp-Himar1; D a random insert ITR-aac(3)IV-ITR with an apramycin resistance gene in the middle;

(7) respectively inserting the three fragments amplified in step (5) into a plasmid pKC1139 used as a skeleton to obtain a plasmid pLRM04;

(8) transducting the plasmid pLRM04 obtained in step (6) into the Streptomyces containing the reporter plasmid obtained in step (3) by conjugation, and verifying;

(9) culturing the Streptomyces strain obtained in step (8), adding hygromycin with a certain concentration in the culturing process to activate the expression of hpAp-Himar1 gene and start the activity of the transposon, and randomly inserting the ITR-aac(3)IV-ITR fragment into the Streptomyces genome to collect a large number of randomly mutated strains;

(10) screening the randomly mutated strains obtained in step (9) with a reporter gene threshold obtained in step (5), and screening out strains with a phenotype higher than the threshold in step (5);

(11) carrying out liquid culture on the Streptomyces strains obtained in step (10), and extracting high-quality genomic DNA, and uniformly breaking the genome into fragments with a certain size;

(12) blunting all ends of the fragments obtained in step (11) using T4 DNA polymerase, and dephosphorylating; and after dephosphorylation, digesting the linearized cosmid with a blunt-end enzyme, and ligating with genome fragments obtained in this step;

(13) coating a ligation product obtained in step (12) with a phage protein, infecting Escherichia coli, and coating on a LB plate with corresponding antibiotics with cosmid resistance and apramycin;

(14) carrying out amplification culture of an Escherichia coli single colony grown on the LB plate in step (13), extracting the cosmid, and sequencing;

(15) according to a sequencing result of step (14), comparing in a Streptomyces genome database by using DNA sequence comparison technology, and accurately determining an insertion position of the random insert ITR-aac(3)IV-ITR in the Streptomyces genome, and determining a destroyed gene in the Streptomyces genome; and

(16) designing a gene knockout scheme, knocking out the gene positioned in step (15), and verifying a regulation mechanism of the gene on the target gene.

In the present invention, the Streptomyces used is Streptomyces for which a stable genetic manipulation can be carried out under laboratory conditions.

The reporter gene system selected in step (2) is a reporter gene system available to Streptomyces of a resistance gene reporter system, a fluorescent protein reporter system and a substrate color development reporter system.

The threshold screened in step (5) corresponds to a corresponding reporter system, the resistance gene reporter system corresponds to an upper limit of an antibiotic concentration, the fluorescent protein reporting system corresponds to a fluorescence display intensity, and the substrate color development reporter system corresponds to a color development intensity.

The cosmid used in step (12) is a cosmid for phage packaging.

The Escherichia coli selected in step (13) is Escherichia coli infected by phage.

A gene knockout system used in step (16) is a knockout system capable of stably knocking out the target gene, including a homologous recombination knockout system, a cosmid knockout system, and a CRISPR/cas9 mediated Streptomyces knockout system.

Compared with the prior art, the present invention has the advantages that:

1) In the present invention, the target gene promoter is screened for negative regulatory factors in Streptomyces, the screening environment is stable, the false positive rate of the obtained negative regulatory factors is low, the subsequent verification work for regulatory factors can be greatly reduced, and the method is efficient, accurate and convenient to operate.

2) In the present invention, the promoter regulatory factors of the target genes are globally screened on the Streptomyces genome, and the screening flux is high, so that the negative regulatory factors of all target genes can be screened theoretically.

3) The present invention is widely used in Streptomyces, and all Streptomyces for which genetic manipulation can be carried out can use the present invention to screen negative regulatory factors of target genes.

4) The present invention can be widely used in the field of screening and reforming industrial Streptomyces metabolites with high yield and good genetic stability, and is suitable for industrial production and applications.

### BRIEF DESCRIPTION OF DRAWINGS

FIG. 1: the CRISPR/cas9 system was used to knock out the phaR gene in Example 1. in the Figure, 1, 2, 3, 4 and 5 denote PCR amplified fragments of the knockout strain;

FIG. 2: the EMSA results of the PhaR protein and dptEp promoter in Example 1;

FIG. 3: the qRT results of the dptE genes of the phaR knockout strain (ΔphaR) and Streptomyces roseosporus L30 (WT) in Example 1; and

FIG. 4: comparison of the yield of daptomycin by shake flask fermentation between the phaR knockout strain (ΔphaR) and Streptomyces roseosporus L30(WT) in Example 1.

### DESCRIPTION OF EMBODIMENTS

The present invention will be further described in detail with reference to the drawings and specific embodiments.

### Example 1

The method of the present invention is used to screen the negative regulatory proteins of the dptE gene in the daptomycin-producing strain, i.e., Streptomyces roseosporus L30. Streptomyces roseosporus L30 is a daptomycin-producing Streptomyces strain industrially. Its genome sequence was also determined and the daptomycin synthesis gene cluster was located. In the whole daptomycin synthetic gene cluster, the direct synthetic protein of daptomycin is encoded by five genes, namely dptE, dptF, dptA, dptBC and dptD. These five genes constitute a cistron, and dptEP, the promoter of the dptE gene, regulates the transcription and translation steps of the whole cistron. Previous studies have shown that high expression of the five genes of dptE, dptF, dptA, dptBC and dptD can effectively improve the industrial fermentation yield of daptomycin. Therefore, we screened and knocked out the negative regulatory factors of the promoter dptEp in the genome of Streptomyces roseosporus L30 by this method, so as to realize the high yield of daptomycin by this strain in the industrial fermentation process. The specific implementation steps are as follows:

(1) the gene dptE (SEQ ID No: 1) in the genome of the daptomycin-producing strain, i.e., the Streptomyces roseosporus L30, was selected and the upstream promoter sequence dptEp (SEQ ID No: 2) of the dptE gene was amplified;

(2) a reporter gene available in Streptomyces roseosporus L30 was selected as a kanamycin resistance gene neo, and a plasmid system of the reporter gene system which can be operated genetically in Streptomyces roseosporus was constructed. With pIJ8660 as the skeleton plasmid, the Apra resistance gene was replaced with Spectinomycin resistance gene Spec at the SacI digestion site, and the kanamycin resistance gene neo was inserted between NdeI and NotI digestion sites.

(3) the promoter sequence dptEp in step (1) was integrated into the reporter plasmid system in step (2) between BamHI and BglII digestion sites upstream the reporter gene;

(4) the reporter plasmid obtained in step (4) was transducted into wild-type Streptomyces roseosporus L30 by conjugation, and was verified;

(5) according to the selected reporter gene system, threshold screening of an expression level of the reporter gene of the Streptomyces strain obtained in the step (4) was carried out; the screening results showed that the highest resistance concentration of kanamycin obtained in step (4) was 300 μg/ml on a YMG plate.

(6) three DNA fragments were amplified: \textcircled{1} hygromycin resistance gene hph; \textcircled{2} thiostrepton-induced promoter and transposon tipAp-Himar1; \textcircled{3} a random insert ITR-aac(3)IV-ITR with an apramycin resistance gene in the middle;

(7) the three fragments amplified in step (5) were respectively inserted into a plasmid pKC1139 used as a skeleton to obtain a plasmid pLRM04;

(8) the plasmid pLRM04 obtained in step (6) was transducted into the Streptomyces containing the reporter plasmid obtained in step (3) by conjugation, and was verified;

(9) the Streptomyces strain obtained in step (8) was cultured, hygromycin with a certain concentration was added in the culturing process to activate the expression of tipAp-Himar1 gene and start the activity of the transposon, and the ITR-aac(3)IV-ITR fragment was randomly inserted into the Streptomyces genome to collect a large number of randomly mutated strains;

(10) the randomly mutated strains obtained in step (9) were screened with a reporter gene threshold obtained in step (5), and strains with a phenotype higher than the threshold in step (5) were screened out;

(11) liquid culture was performed on the high kanamycin resistance strains obtained in step (10), and a genome with improved quality was extracted, and uniformly broken into fragments with a size of about 40 Kb;

(12) all ends of the fragments obtained in step (11) were filled using T4 DNA polymerase, and dephosphorylated; and after dephosphorylation, cosmid pHAQ31 was linearized with restriction enzyme NheI and then dephosphorylated, and then cut into two segments with StuI enzyme to separate two cos sites; the genome fragments obtained in this step were ligated with each other by a T4 ligase;

(13) a ligation product obtained in step (12) was coated with a phage protein, infected with Escherichia coli DH10B, and was coated on a LB plate with corresponding antibiotics with cosmid resistance and apramycin;

(14) liquid amplification culture of the Escherichia coli single colony grown on the LB plate in step (13) was carried out, the cosmid was extracted and subjected to sequencing;

(15) according to a sequencing result of step (14), comparison was carried out in a Streptomyces genome database by using DNA sequence comparison technology, and the insertion position of the random insert ITR-aac(3)IV-ITR in the Streptomyces genome was accurately determined; and the gene mutated by insertion was identified as phaR (SEQ ID No: 3);

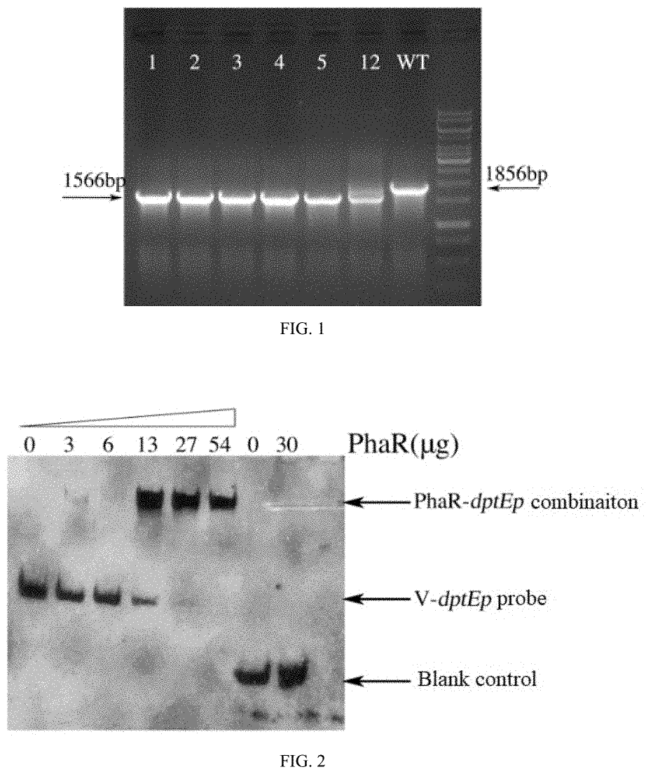

(16) a CRISPR/cas9 mediated gene knockout scheme was designed to knock out the phaR gene (FIG. 1).

(17) a purified PhaR protein was expressed in vitro, and PhaR protein and dptEp were verified to be combined with each other by an EMSA experiment (FIG. 2);

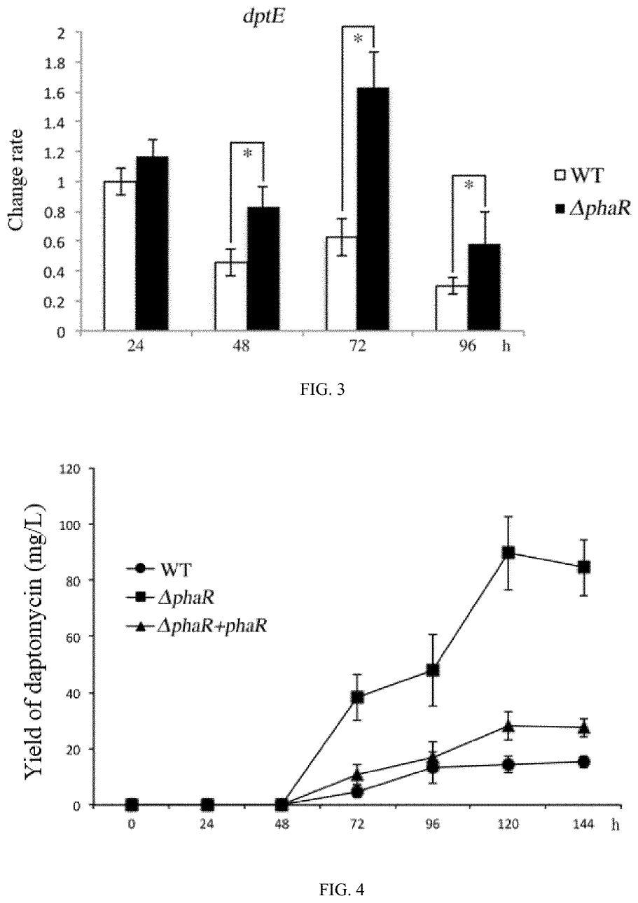

(18) the phaR gene knockout strain and Streptomyces roseosporus L30 were cultured in liquid, then RNA was extracted and analyzed by fluorescence quantitative PCR; the results showed that the expression of the dptE gene in the phaR knockout strain was 2-3 times higher than that in wild-type Streptomyces roseosporus (FIG. 3).

(19) the phaR gene knockout strain obtained in step (16) and Streptomyces roseosporus L30 were fermented (see table 1 for fermentation conditions);

(20) the fermentation product of the phaR gene knockout strain and Streptomyces roseosporus L30 were subjected to HPCL detection (see FIG. 4 for detection results); the fermentation results of the two strains showed that the yield of daptomycin in the phaR knockout strain increased obviously, which further proved the high expression of daptomycin synthetic gene cluster in the phaR knockout strain.

The Streptomyces roseosporus L30 is preserved in the China General Microbiological Culture Collection Center with a preservation number of CGMCC No. 15745 and a preservation date of May 9, 2018 at No. 3, Courtyard 1, Beichen West Road, Chaoyang District, Beijing.

The above experimental results prove that the PhaR protein is a negative regulator for the dptE gene, and dptE and its downstream genes are highly expressed in its gene knockout strain, thereby proving the effectiveness of the invention.

TABLE 1
Fermentation process of Streptomyces roseosporus in Example 1

| | |
|---|---|
| Seed medium | a liquid medium of 2% TSB and 5% PEG6000 |
| Seed culture | 30 ml medium /250 ml container, 30° C., |
| process | 22-26 h, 250 rpm |
| fermentation | 0.3% of yeast extract, 0.3% of malt extract, 0.5% |
| medium | of tryptone and 4% of glucose |
| fermentation | 30 ml/250 ml, 30° C., 144-168 h, 250 rpm |
| culture process | |
| Supplementary | decanoic acid was added at a volume ratio |
| feeding process | of 1/1000 every time twice a day after |
| | 36 hours of fermentation |

1. A screening method of negative regulatory factors of a Streptomyces biosynthesis gene cluster, comprising:
constructing a reporter system mediated by a promoter of a self-owned target gene in a Streptomyces cell, and then randomly mutating Streptomyces with the reporter system by using a random mutation system constructed based on a transposon Himar1;
intensively screening Streptomyces strains that have been subjected to random mutation to obtain a Streptomyces strain with high expression of the target gene;
packaging a genome of the Streptomyces strain with high expression of the target gene by a phage packaging method and screening out a cosmid with a random insert; and
finally determining an accurate position of the random insert in the genome of the Streptomyces strain with high expression of the target gene by sequencing DNAs of the cosmid.

2. The screening method of negative regulatory factors of a Streptomyces biosynthesis gene cluster according to claim 1, wherein the method comprises the following specific steps:
(1) selecting a target gene, which needs to be screened for a regulatory factor, in a Streptomyces genome, and amplifying an upstream promoter sequence of the target gene;
(2) selecting an available reporter gene system in the Streptomyces, constructing a plasmid system in which the reporter gene system can be genetically operated in the Streptomyces, and determining that there is no promoter upstream of a reporter gene in the plasmid system;
(3) integrating the promoter sequence in step (1) into a reporter plasmid system in step (2) upstream of the reporter gene;
(4) transducting a reporter plasmid obtained in step (3) into wild-type Streptomyces by conjugation, and verifying;
(5) according to the selected reporter gene system, performing threshold screening of an expression level of the reporter gene of the Streptomyces strain obtained in the step (4);
(6) amplifying three DNA fragments: \textcircled{1} a hygromycin resistance gene hph; \textcircled{2} hygromycin-induced promoter and transposon tipAp-Himar1; \textcircled{3} a random insert ITR-aac(3)IV-ITR with an apramycin resistance gene in the middle;
(7) respectively inserting the three fragments amplified in step (5) into a plasmid pKC1139 used as a skeleton to obtain a plasmid pLRM04;
(8) transducting the plasmid pLRM04 obtained in step (6) into the Streptomyces containing the reporter plasmid obtained in step (3) by conjugation, and verifying;
(9) culturing the Streptomyces strain obtained in step (8), adding hygromycin with a certain concentration in the culturing process to activate expression of tipAp-Himar1 gene and start activity of the transposon, and randomly inserting the ITR-aac(3)IV-ITR fragment into the Streptomyces genome to collect a large number of randomly mutated strains;
(10) screening the randomly mutated strains obtained in step (9) with a reporter gene threshold obtained in step (5), and screening out strains with a phenotype higher than the threshold in step (5);
(11) carrying out liquid culture on the Streptomyces strains obtained in step (10), and extracting a genome with high quality, and uniformly breaking the genome into fragments with a certain size;
(12) blunting all ends of the fragments obtained in step (11) using T4 DNA polymerase, and dephosphorylating; and after dephosphorylation, digesting the linearized cosmid with a blunt-end enzyme, and ligating with genome fragments obtained in this step;
(13) coating a ligation product obtained in step (12) with a phage protein, infecting Escherichia coli, and coating on a LB plate with corresponding antibiotics with cosmid resistance and apramycin;
(14) carrying out amplification culture of an Escherichia coli single colony grown on the LB plate in step (13), extracting the cosmid, and sequencing;
(15) according to a sequencing result of step (14), comparing in a Streptomyces genome database by using DNA sequence comparison technology, and accurately determining an insertion position of the random insert ITR-aac(3)IV-ITR in the Streptomyces genome, and determining a destroyed gene in the Streptomyces genome; and
(16) designing a gene knockout scheme, knocking out the gene positioned in step (15), and verifying a regulation mechanism of the gene on the target gene.

3. The screening method of negative regulatory factors of a Streptomyces biosynthesis gene cluster according to claim 2, wherein the Streptomyces used is Streptomyces for which a stable genetic manipulation can be carried out under laboratory conditions.

4. The screening method of negative regulatory factors of a Streptomyces biosynthesis gene cluster according to claim 2, wherein the reporter gene system selected in step (2) is a reporter gene system available to Streptomyces including a resistance gene reporter system, a fluorescent protein reporter system and a substrate color development reporter system.

5. The screening method of negative regulatory factors of a Streptomyces biosynthesis gene cluster according to claim 4, wherein the threshold screened in step (5) corresponds to a corresponding reporter system, the resistance gene reporter system corresponds to an upper limit of an antibiotic concentration, the fluorescent protein reporter system corresponds to a fluorescence display intensity, and the substrate color development reporter system corresponds to a color development intensity.

6. The screening method of negative regulatory factors of a Streptomyces biosynthesis gene cluster according to claim 2, wherein the cosmid used in step (12) is a cosmid for phage packaging.

7. The screening method of negative regulatory factors of a Streptomyces biosynthesis gene cluster according to claim 2, wherein the Escherichia coli selected in step (13) is Escherichia coli infected by phage.

8. The screening method of negative regulatory factors of a Streptomyces biosynthesis gene cluster according to claim 2, wherein a gene knockout system used in step (16) is a knockout system capable of stably knocking out the target gene, including a homologous recombination-mediated knockout system, a cosmid-mediated knockout system, and a CRISPR/cas9-mediated Streptomyces knockout system.
